(19) United States
(12) Patent Application Publication
(10) Pub. No.: US 2025/0388705 A1
(43) Pub. Date: Dec. 25, 2025

(54) WATER-SOLUBLE, BIODEGRADABLE DIHYDROXYALKYL CELLULOSE

(71) Applicant: ISP Investments LLC, Wilmington, DE (US)

(72) Inventors: Fangbin ZHOU, Hockessin, DE (US); MICHAEL FRANZKE, Barendrecht (NL); Edward Di ANTONIO, Staten Island, NY (US); Gijsbert KROON, Giessenburg (NL)

(73) Assignee: ISP Investments LLC, Wilmington, DE (US)

(21) Appl. No.: 19/107,835

(22) PCT Filed: Aug. 29, 2023
(86) PCT No.: PCT/US2023/031414

§ 371 Date: Feb. 28, 2025

Related U.S. Application Data
(60) Provisional application No. 63/402,273, filed on Aug. 30, 2022.

(51) Int. Cl. C08B 11/02; A61K 8/73; A61Q 5/00; A61Q 19/00; A61Q 19/10

(52) U.S. Cl. CPC C08B 11/02; A61K 8/731; A61Q 5/00; A61Q 19/00; A61Q 19/10

(57) ABSTRACT

The present application provides a water-soluble, partially biodegradable dihydroxyalkyl cellulose having about 0.7 to about 2.0 units of dihydroxyalkyl moiety per anhydroglucose moiety, having (i) a molecular weight of dihydroxyalkyl cellulose is in the range of from about 10,000 Daltons to about 1,500,000 Daltons, (ii) a biodegradation rate of ≥3% in an aquatic medium, and (iii) a water solubility at a threshold level of 2 g/L. Also discloses a method for preparing dihydroxyalkyl cellulose and functionalized dihydroxyalkyl cellulose compounds and their use in personal care applications.

### FIELD OF THE INVENTION

The presently disclosed process(es), procedure(s), method(s), product(s), result(s), and/or concept(s) (collectively referred to hereinafter as the “present application, present disclosure or invention”) relates generally to a water-soluble, partially biodegradable dihydroxyalkyl cellulose, method of producing the dihydroxyalkyl cellulose, and its uses thereof.

### BACKGROUND OF THE INVENTION

This invention relates to a water-soluble, partially biodegradable dihydroxyalkyl cellulose, particularly it relates to a water-soluble, partially biodegradable dihydroxyalkyl cellulose having about 0.7 to about 2.0 units of dihydroxyalkyl moiety per anhydroglucose moiety.

European Chemicals Agency (ECHA) proposed a wide-ranging restriction on microplastics in products placed on the EU/EEA market to avoid or reduce their release to the environment. As the global companies are targeting to eliminate all ‘avoidable’ microplastics, the new trends are, ‘alternative plastics’, such as biobased, biodegradable and compostable plastics. Further, chemically modified natural polymers generate the huge share in the market. In particular modified cellulose, modified other polysaccharides and other modified natural polymers are key market drivers in biodegradable polymers with many applications in health care, oil field, personal care, electronics, and homecare.

Since the initial work of Lilienfeld and of Dreyfus in the 1920's relating to cellulose ethers, a continuing quest has gone on for new derivatives of cellulose and for modification of those already known. Out of this research, carboxymethyl cellulose, hydroxyethyl cellulose, methyl cellulose, hydroxypropyl cellulose and ethyl cellulose have developed into very significant items of commerce.

Among the early researches into the preparation of cellulose ethers, it was suggested that cellulose can be reacted with 3-chloro-1,2-propanediol to form ethers. See, e.g., Dreyfus, U.S. Pat. No. 1,502,379, and Lilienfeld, U.S. Pat. No. 1,722,927. Both of these patents prepare what is referred to as an “oxyalkyl derivative of cellulose” by reaction of alkali cellulose with the above-mentioned 3-chloro-1,2-propanediol. In each case, however, little is told of the properties of the product except that it is soluble in alkali or organic liquids but insoluble in water. Nothing is stated in either reference concerning the substitution levels of these products.

At a later date in U.S. Pat. No. 2,572,039 to Klug et al, relates to the manufacture of cellulose ethers and particularly to an improvement in the manufacture of hydroxyalkyl ethers of cellulose. Various methods have been proposed and utilized for the preparation of hydroxyalkyl derivatives of cellulose and other carbohydrates. Generally, the processes heretofore proposed have been found deficient in simplicity and economy and the products obtained have lacked desired uniformity and desired solubility.

Canadian patent publication No. 1095029A discloses a reaction product of a cellulose ether and about 0.05 to 10 parts by weight, per part of cellulose ether, of a reactant selected from the group consisting of borate salts, antimony salts and easily saponifiable esters of boric acid, the cellulose ether being characterized by (a) having as sole substituent on the cellulose about 1.4 to 6 dihydroxypropyl units per anhydro-glucose unit, (b) being soluble in water and (c) being thermoplastic.

U.S. Pat. No. 9,782,609 discloses dihydroxyalkyl substituted galactomannan polymers. More particularly, but not by way of limitation, disclosed and/or claimed inventive concept(s) further relates to optionally modifying the substituted galactomannan polymers with cationic and/or hydrophobic moieties.

U.S. Pat. No. 4,096,326 discloses an ether of cellulose having at least about 1.4 dihydroxypropyl units per anhydroglucose unit, said ether of cellulose being characterized by being soluble in water and by being thermoplastic.

In view of the foregoing, still there is a need for a water-soluble, partially biodegradable dihydroxyalkyl cellulose having better biodegradable and solubility properties.

Surprisingly, it was found out that dihydroxyalkyl cellulose having about 0.7 to about 2.0 units of dihydroxyalkyl moiety per anhydroglucose moiety has water-solubility and partially biodegradability properties.

Accordingly, the present invention relates to a water-soluble, partially biodegradable dihydroxyalkyl cellulose having about 0.7 to about 2.0 units of dihydroxyalkyl moiety per anhydroglucose moiety.

### SUMMARY OF THE INVENTION

The primary aspect of the present application is to provide a water-soluble, partially biodegradable dihydroxyalkyl cellulose having about 0.7 to about 2.0 units of dihydroxyalkyl moiety per anhydroglucose moiety.

Another aspect of the present application provides a dihydroxyalkyl cellulose having about 0.7 to about 2.0 units of dihydroxyalkyl moiety per anhydroglucose moiety, having a biodegradation rate of ≥3% in an aquatic medium when measured by a method of Organisation for Economic Co-operation and Development (OECD) Test number 301 or 302, and meets the standards of water solubility method of OECD Test number 105 at a threshold level of 2 g/L.

Another non-limiting aspect of the present application discloses a dihydroxyalkyl cellulose with at least one anionic, cationic or hydrophobic functional moiety.

Yet another aspect of the present application discloses a method for preparing water-soluble, partially biodegradable dihydroxyalkyl cellulose.

Another aspect of the present application provides a method for preparing water-soluble, partially biodegradable dihydroxyalkyl cellulose with at least one anionic, cationic or hydrophobic functional moiety.

Another aspect of the present application provides a composition comprising a water-soluble, partially biodegradable dihydroxyalkyl cellulose having about 0.7 to about 2.0 units of dihydroxyalkyl moiety per anhydroglucose moiety and one or more ingredients.

One more aspect of the present application discloses a personal care composition comprising: (a) from about 0.1 to about 20% (w/w) of the water-soluble, partially biodegradable dihydroxyalkyl cellulose, and (b) from about 80 to about 99.9% (w/w) of one or more personal care ingredient.

### DETAILED DESCRIPTION OF THE INVENTION

Before explaining at least one aspect of the disclosed and/or claimed inventive concept(s) in detail, it is to be understood that the disclosed and/or claimed inventive concept(s) is not limited in its application to the details of construction and the arrangement of the components or steps or methodologies set forth in the following description or illustrated in the drawings. The disclosed and/or claimed inventive concept(s) is capable of other aspects or of being practiced or carried out in various ways. Also, it is to be understood that the phraseology and terminology employed herein is for the purpose of description and should not be regarded as limiting.

As utilized in accordance with the disclosure, the following terms, unless otherwise indicated, shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular.

The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out the invention.

All percentages, parts, proportions and ratios as used herein, are by weight of the total composition, unless otherwise specified. All such weights as they pertain to listed ingredients are based on the active level and, therefore; do not include solvents or by-products that may be included in commercially available materials, unless otherwise specified.

All publications, articles, papers, patents, patent publications, and other references cited herein are hereby incorporated herein in their entirety for all purposes to the extent consistent with the disclosure herein.

The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached.

In addition, the quantities of 100/1000 are not to be considered limiting as lower or higher limits may also produce satisfactory results.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “each independently selected from the group consisting of” means when a group appears more than once in a structure, that group may be selected independently each time it appears.

As used herein, the term “OECD test” refers to testing standards and methods set and validated by the Organization of Economic Cooperation and Development (OECD).

As used herein, the term “water-soluble” refers to water-soluble rate measured by a method of OECD Test number 105, wherein the method provides solubility at a threshold level of 2 g/L.

As used herein, the term “partially biodegradable” refers to biodegradation rate in an aquatic medium when measured by a method of OECD Test number 301 or 302.

As used herein, the term “cellulose” refers to a homopolymer of B (1→4) linked D-glucose units that form a linear chain and has the following structure:

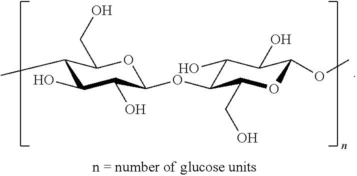

Cellulose can contain several hundred to several thousand or more glucose units, making cellulose a polysaccharide. Cellulose is found in many natural products, such as the cell walls of plants, and thus can be found in wood, pulp and cotton, among others.

The term “β(1→4)” or “beta-1,4-glucosidic linkage” as used herein refers to the covalent bond that joins glucose molecules to each other through carbons 1 and 4 on adjacent glucose monomers in a glucan.

The term “glucan” herein refers to a polysaccharide of D-glucose monomers that are linked by glucosidic linkages, which are a type of glycosidic linkage.

The terms “glycosidic linkage”, “glycosidic bond” and the like are used interchangeably herein and refer to the covalent bond that joins a carbohydrate molecule to another carbohydrate molecule. The terms “glucosidic linkage”, “glucosidic bond” and the like are used interchangeably herein and refer to a glycosidic linkage between two glucose molecules in a glucan.

As used herein, the term “saccharide” refers to a sugar, such as a monosaccharide, a disaccharide, an oligosaccharide or a polysaccharide. Monosaccharides include, but are not limited to, glucose, ribose and fructose. Disaccharides include, but are not limited to, sucrose and lactose. Polysaccharides include, but are not limited to, cellulose, hemicellulose and lignocellulose or starch. Other saccharides are useful in the scope of present invention.

In a non-limiting embodiment, the present application is to provide a water-soluble, partially biodegradable dihydroxyalkyl cellulose having about 0.7 to about 2.0 units of dihydroxyalkyl moiety per anhydroglucose moiety.

According to another embodiment, the present application relates to a dihydroxyalkyl cellulose, wherein the dihydroxyalkyl cellulose having about 1.0 to about 1.7 units of dihydroxyalkyl moiety per anhydroglucose moiety.

According to one more embodiment, the dihydroxyalkyl cellulose having about 0.7 to about 0.8 units, about 0.9 to about 1.0 units, about 1.1 to about 1.2 units, about 1.3 to about 1.4 units, about 1.5 to about 1.6 units, about 1.7 to about 1.8 units, about 1.9 to about 2.0 units of dihydroxyalkyl moiety per anhydroglucose moiety.

According to one more embodiment, the molecular weight of dihydroxyalkyl cellulose is in the range of from about 10,000 Daltons to about 1,500,000 Daltons.

In a non-limiting embodiment, the molecular weight of dihydroxyalkyl cellulose is in the range of from about 40,000 Daltons to about 1,200,000 Daltons.

According to another embodiment, the molecular weight of dihydroxyalkyl cellulose is in the range of from about 10,000 Daltons to about 20,000 Daltons, from about 20,000 Daltons to about 30,000 Daltons, from about 30,000 Daltons to about 40,000 Daltons, from about 40,000 Daltons to about 50,000 Daltons, from about 50,000 Daltons to about 60,000 Daltons, from about 60,000 Daltons to about 70,000 Daltons, from about 70,000 Daltons to about 80,000 Daltons, from about 80,000 Daltons to about 90,000 Daltons, and from about 90,000 Daltons to about 100,000 Daltons.

According to another embodiment, the molecular weight of dihydroxyalkyl cellulose is in the range of from about 100,000 Daltons to about 200,000 Daltons, from about 200,000 Daltons to about 300,000 Daltons, from about 300,000 Daltons to about 400,000 Daltons, from about 400,000 Daltons to about 500,000 Daltons, from about 500,000 Daltons to about 600,000 Daltons, from about 600,000 Daltons to about 700,000 Daltons, from about 700,000 Daltons to about 800,000 Daltons, from about 800,000 Daltons to about 900,000 Daltons, and from about 900,000 Daltons to about 1000,000 Daltons.

According to another embodiment, the molecular weight of dihydroxyalkyl cellulose is in the range of from about 10,00,000 Daltons to about 11,00,000 Daltons, from about 11,00,000 Daltons to about 12,00,000 Daltons, from about 12,00,000 Daltons to about 13,00,000 Daltons, from about 13,00,000 Daltons to about 14,00,000 Daltons, from about 14,00,000 Daltons to about 15,00,000 Daltons.

According to one more embodiment, the dihydroxyalkyl cellulose is selected from the group consisting of dihydroxyethyl cellulose, dihydroxypropyl cellulose, dihydroxybutyl cellulose, and dihydroxypentyl cellulose.

According to one more embodiment, the dihydroxyalkyl cellulose is dihydroxypropyl cellulose.

According to another embodiment, the dihydroxyalkyl cellulose shows a partial biodegradation rate of ≥3% in an aquatic medium when measured by OECD Test number 301 or 302.

According to one more embodiment, it is contemplated that the dihydroxyalkyl cellulose shows a biodegradation rate of ≥3%, ≥5%, ≥10%, ≥15%, ≥20%, ≥25%, ≥30%, ≥35%, ≥ 40%, ≥45%, ≥50%, ≥55%, ≥60%, ≥65%, ≥70%, ≥75%, or ≥80% in an aquatic medium when measured by OECD Test method number 301 or 302.

According to one more embodiment, the dihydroxyalkyl cellulose has a water-solubility that meets the threshold value of 2 g/liter as per OECD Test method number 105.

According to one more embodiment, the dihydroxyalkyl cellulose is functionalized with at least one anionic, cationic or hydrophobic functional moiety.

According to another non-limiting embodiment, the anionic functional moiety is an alkenyl succinic anhydride. According to one non-limiting embodiment, the alkenyl succinic anhydride is selected from the group consisting of octenyl succinic anhydride, dodecenyl succinic anhydride, and hexacenyl succinic anhydride.

According to another embodiment, the cationic functional moiety is selected from the group consisting of glycidyl trimethyl ammonium chloride, glycidyl trimethyl ammonium bromide, glycidyl trimethyl ammonium iodide, glycidyl trimethyl ammonium sulfate, glycidyl trimethyl ammonium acetate, glycidyl triethyl ammonium chloride, glycidyl triethyl ammonium bromide, glycidyl tripropyl ammonium chloride, glycidyl tributyl ammonium chloride, glycidyl triisopropyl ammonium chlorides, N-3,4-epoxybutyl trimethyl ammonium chloride, N-3,4-epoxybutyl triethyl ammonium bromide, N-4,5-epoxyamyl trimethyl ammonium chloride, dimethylamino-2,3-epoxypropane, diethylamino-2,3-epoxy-propane, dibutylamino-2,3-epoxy-propane, methyl ethyl amino-2,3-epoxypropane, diethylamino-3,4-epoxybutane, dimethylamino-4,5-epoxypentane), and 3-chloro-2-hydroxypropyltrimethylammonium chloride.

According to one more embodiment, the hydrophobic functional moiety is one or more C₄-C₂₂ alkyl radical.

According to one non-limiting embodiment, the C₄-C₂₂ alkyl radical is selected from the group consisting of C₄-C₂₂ alkyl dihydroxyalkyl cellulose, C₄-C₂₂ 3-hydroxylalkyl dihydroxyalkyl cellulose, C₄-C₂₂ alkyl succinated dihydroxyalkyl cellulose, C₄-C₂₂ alkenyl dihydroxyalkyl cellulose, and C₄-C₂₂ alkonic esterified dihydroxyalkyl cellulose, C₄-C₂₂ alkyl cationic dihydroxyalkyl cellulose and combinations thereof.

According to one more embodiment, the water-soluble, partially biodegradable dihydroxyalkyl cellulose is prepared by a method, comprising the steps of: (a) alkalizing a cellulose with an aqueous caustic solution in a temperature range from about at 10° C. to about 50° C.; (b) adding a diol compound or oxiranylalkanol compound to the resultant of step (a) and heating the resultant in a temperature ranging from about 50° C. to about 150° C. for a period of from about 1 to about 8 hours to obtain a low substituted dihydroxyalkyl cellulose; (c) cooling the resultant of step (b) to a temperature ranging from about 20° C. to about 60° C. and adding an additional amount of caustic solution and diol compound; (d) heating the resultant of step (c) to a temperature ranging from about 50° C. to about 150° C. for a period of about 1 hour to about 8 hours to obtain desired substituted dihydroxyalkyl cellulose, wherein optionally the diol and aqueous caustic solution are added; (e) cooling the resultant of step (d) to a temperature of from about 20° C. to about 40° C. for a period of about 1 hour to about 3 hours and adjusting the pH to about 5.5 to about 8.5; and (f) filtering, washing, drying and grinding the resultant of step (e) to obtain desired dihydroxyalkyl cellulose.

According to another embodiment, the method for preparing cationic, anionic, or hydrophobic functional dihydroxyalkyl cellulose, comprising the steps of: a) mixing from about 5% (w/w) to 45% (w/w) of dihydroxyalkyl cellulose, from about 20% (w/w) to 70% (w/w) of IPA, from about 5% (w/w) to 50% (w/w) of water and from about 1% (w/w) to 15% (w/w) of caustic in a flask and stirring from about 0.1 hour to 1 hour; b) adding from about 1% (w/w) to 30% (w/w) of cationic, anionic, or hydrophobic agent to the resultant of (a) and heat the mixture to a temperature ranging from about 30° C. to 120° C. for about 1 to about 16 hours; and c) lower the temperature of the resultant of (b) to room temperature, neutralize the resultant with 50% (w/w) to 100% (w/w) of acid, and wash with different strength of 50% (w/w) to 100% (w/w) of IPA, acetone, or methanol; and d) dry the resultant in temperature ranging from about 30° C. to about 80° C. in an oven to retrieve cationic, anionic, or hydrophobic functional dihydroxyalkyl cellulose. According to another embodiment, the cellulose is selected from the group consisting of wood pulps, and cotton linters.

According to another embodiment, the caustic solution is prepared from the group consisting of sodium hydroxide, potassium hydroxide, calcium hydroxide, sodium carbonate, potassium carbonate, sodium bicarbonate, and potassium bicarbonate.

According to another embodiment, the diol compound is selected from the group consisting of oxiranylalkanol, glycerol monohalohydrin and combinations thereof.

According to one more embodiment, the glycerol monohalohydrin is selected from the group consisting of 3-chloro-1,2-propanediol, 3-bromo-1,2-dihydroxypropanediol, 3-iodo-1,2-dihydroxypropanediol, 3-tosyl-1,2-dihydroxypropanediol, and combinations thereof.

According to one non-limiting embodiment, the oxiranylalkanol compound is selected from the group consisting of oxiranylmethanol (glycidol), oxiranylethanol, oxiranylpropanol, oxanylbutanol, oxanylpentanol, 2-(1-Ethoxyethoxy)-2-(2-oxiranyl) ethanol, and combinations thereof.

According to another embodiment of the present application, the invention relates to a personal care composition comprising: (a) from about 0.1 to about 20% (w/w) water-soluble, partially biodegradable dihydroxyalkyl cellulose of claim invention, and (b) from about 80 to about 99.9% (w/w) of one or more personal care ingredient.

According to one more embodiment, the personal composition is a hair care composition, a skin care composition, a shampoo composition, an oral care composition or a bodywash composition.

According to another embodiment, the personal care ingredient is selected from the group consisting of surfactants, fatty acid soap, hair and skin conditioning agents, suspending aids, emollients, emulsifiers, rheology modifiers, thickening agents, vitamins, hair growth promoters, self-tanning agents, sunscreens, skin lighteners, anti-aging compounds, anti-wrinkle compounds, anti-cellulite compounds, anti-acne compounds, anti-dandruff agents, antiperspirant agents, deodorant agents, hair fixatives, particulates, abrasives, moisturizers, antioxidants, keratolytic agents, anti-static agents, foam boosters, hydrotropes, solubilizing agents, pH adjusting agents, chelating agents, buffering agents, botanicals, hair colorants, hair dye, oxidizing agents, reducing agents, hair and skin bleaching agents, pigments, anticaries, anti-tartar agents, anti-plaque agents, solvents, and combinations thereof. According to another embodiment of the present application, the invention relates to a hair care composition comprising: (a) from about 0.1 to about 20% (w/w) water-soluble, partially biodegradable dihydroxyalkyl cellulose, and (b) from about 80 to about 99.9% (w/w) of one or more hair care ingredient.

According to another embodiment of the present application, a bodywash composition comprising: (a) from about 0.1 to about 10% (w/w) water-soluble, partially biodegradable dihydroxyalkyl cellulose, and (b) from about 90 to about 99.9% (w/w) of one or more ingredient.

Further, certain aspects of the present application are illustrated in detail by way of the following examples. The examples are given herein for illustration of the application and are not intended to be limiting thereof.

### EXAMPLES

Example 1: Cellulose (45 g) is alkalized with an aqueous/IPA caustic solution (2.5%; 600 g) at a temperature of 20° C. Further, a diol compound or oxiranylalkanol compound (35 g) is added to the resultant of above step and the resultant is heated at a temperature of 60° C. for 1 hours to obtain a low substituted dihydroxyalkyl cellulose. The low substituted dihydroxyalkyl cellulose is cooled to a temperature of 40° C. and then an additional amount of caustic solution (10.2 g caustic) and diol compound (60 g) are added. The above resultant is heated to a temperature 60° C. for an hour to obtain desired substituted dihydroxyalkyl cellulose. The obtained dihydroxyalkyl cellulose is cooled to a temperature 30° C. for a period of one hour and the pH of the above reactants is adjusted to about 5.5 to about 8.5. The obtained dihydroxyalkyl cellulose is filtered, washed, dried and ground to obtain desired dihydroxyalkyl cellulose.

TABLE 1
Dihydroxyalkyl cellulose with different units of
dihydroxyalkyl moiety per anhydroglucose moiety

| | | | | % |
|---|---|---|---|---|
| | | Unsub. | | biodegradation |
| Example | DHP-MS | % | MW | (28 day) |
| Example 1 | 1.09 | 38.7 | 839000 | 28% |
| Example 2 | 1.48 | 30.8 | 981000 | 26% |

Example 2: Example 1 is repeated by changing the amount of the chemicals as described in Table 1 to obtain Dihydroxyalkyl cellulose of Example 2.

Biodegradability test (OECD Test number 301): This Test Guideline describes six methods that permit the screening of chemicals for ready biodegradability in an aerobic aqueous medium.

OECD test number 302: This Test Guideline describes six methods that permit the screening of chemicals for ready biodegradability in an aerobic aqueous medium.

Solubility test (OECD Test number 105): This Test Guideline describes methods to determine the water solubility of test substances.

Molecular weight determination: weight average molecular weight of each sample was measured by using gel permeation chromatography.

While the compositions and methods of the disclosed and/or claimed inventive concept(s) have been described in terms of particular aspects, it will be apparent to those of ordinary skill in the art that variations may be applied to the compositions and/or methods and in the steps or in the sequence of steps of the method described herein without departing from the concept, spirit and scope of the disclosed and/or claimed inventive concept(s). All such similar substitutes and modifications apparent to those skilled in the art are deemed to be within the spirit, scope and concept of the disclosed! and/or claimed inventive concept(s).

What is claimed is:

1. A water-soluble, partially biodegradable dihydroxyalkyl cellulose having about 0.7 to about 2.0 units of dihydroxyalkyl moiety per anhydroglucose moiety.

2. The dihydroxyalkyl cellulose according to claim 1, wherein the dihydroxyalkyl cellulose having about 1.0 to about 1.7 units of dihydroxyalkyl moiety per anhydroglucose moiety.

3. The dihydroxyalkyl cellulose according to claim 1, wherein the molecular weight of dihydroxyalkyl cellulose is in the range of from about 10,000 Daltons to about 1,500,000 Daltons.

4. The dihydroxyalkyl cellulose according to claim 1, wherein the molecular weight of dihydroxyalkyl cellulose is in the range of from about 40,000 Daltons to about 1,200,000 Daltons.

5. The dihydroxyalkyl cellulose according to claim 1, wherein the dihydroxyalkyl cellulose is selected from the group consisting of dihydroxyethyl cellulose, dihydroxypropyl cellulose, dihydroxybutyl cellulose, and dihydroxypentyl cellulose.

6. The dihydroxyalkyl cellulose according to claim 1, wherein the dihydroxyalkyl cellulose is dihydroxypropyl cellulose.

7. The dihydroxyalkyl cellulose according to claim 1, wherein the dihydroxyalkyl cellulose shows a partial biodegradation rate of ≥3% in an aquatic medium when measured by a method of OECD Test number 301 or 302.

8. The dihydroxyalkyl cellulose according to claim 1, wherein the dihydroxyalkyl cellulose shows evidence of biodegradation within 60 days, or within 28 days.

9. The dihydroxyalkyl cellulose according to claim 1, wherein the dihydroxyalkyl cellulose meets the standards of water solubility method OECD Test number 105 at a threshold level of 2 g/L.

10. The dihydroxyalkyl cellulose according to claim 1, wherein the dihydroxyalkyl cellulose is further functionalized with at least one anionic, cationic or hydrophobic functional moiety.

11. The dihydroxyalkyl cellulose according to claim 10, wherein the anionic functional moiety is an alkenyl succinic anhydride.

12. The dihydroxyalkyl cellulose according to claim 11, wherein the alkenyl succinic anhydride is selected from the group consisting of octenyl succinic anhydride, dodecenyl succinic anhydride, and hexacenyl succinic anhydride.

13. The dihydroxyalkyl cellulose according to claim 10, wherein the cationic functional moiety is selected from the group consisting of glycidyl trimethyl ammonium chloride, glycidyl trimethyl ammonium bromide, glycidyl trimethyl ammonium iodide, glycidyl trimethyl ammonium sulfate, glycidyl trimethyl ammonium acetate, glycidyl triethyl ammonium chloride, glycidyl triethyl ammonium bromide, glycidyl tripropyl ammonium chloride, glycidyl tributyl ammonium chloride, glycidyl triisopropyl ammonium chlorides, N-3,4-epoxybutyl trimethyl ammonium chloride, N-3,4-epoxybutyl triethyl ammonium bromide, N-4,5-epoxyamyl trimethyl ammonium chloride, dimethylamino-2,3-epoxypropane, diethylamino-2,3-epoxy-propane, dibutylamino-2,3-epoxy-propane, methyl ethyl amino-2,3-epoxypropane, diethylamino-3,4-epoxybutane, dimethylamino-4,5-epoxypentane), and 3-chloro-2-hydroxypropyltrimethylammonium chloride.

14. The dihydroxyalkyl cellulose according to claim 10, wherein the hydrophobic functional moiety is one or more C₄-C₂₂ alkyl radical.

15. The dihydroxyalkyl cellulose according to claim 14, wherein the hydrophobic functional moiety is selected from the group consisting of C₄-C₂₂ alkyl dihydroxyalkyl cellulose, C₄-C₂₂ 3-hydroxylalkyl dihydroxyalkyl cellulose, C₄-C₂₂ alkyl succinated dihydroxyalkyl cellulose, C₄-C₂₂ alkenyl dihydroxyalkyl cellulose, and C₄-C₂₂ alkonic esterified dihydroxyalkyl cellulose, C₄-C₂₂ alkyl cationic dihydroxyalkyl cellulose and combinations thereof.

16. A method for preparing water-soluble, partially biodegradable dihydroxyalkyl cellulose of claim 1, comprising the steps of:
a) alkalizing a cellulose with an aqueous caustic solution in a temperature;
b) adding a diol compound or oxiranylalkanol compound to the resultant of step (a) and heating the resultant in a temperature ranging from about 50° C. to about 150° C. for a period of from about 1 to about 8 hours to obtain a low substituted dihydroxyalkyl cellulose;
c) cooling the resultant of step (b) to a temperature ranging from about 20° C. to about 60° C.;
d) heating the resultant of step (c) to a temperature ranging from about 50° C. to about 150° C. for a period of about 1 hour to about 8 hours to obtain desired substituted dihydroxyalkyl cellulose, wherein optionally the diol and aqueous caustic solution are added;
e) cooling the resultant of step (d) to a temperature of from about 20° C. to about 40° C. for a period of about 1 hour to about 3 hours and adjusting the pH to about 5.5 to about 8.5; and
f) filtering, washing, drying and grinding the resultant of step (e) to obtain desired dihydroxyalkyl cellulose of claim 1.

17. A method for preparing functionalized dihydroxyalkyl cellulose, comprising the steps of:
a) mixing dihydroxyalkyl cellulose, isopropyl alcohol (IPA), water and caustic in a flask and stirring from about 0.1 hour to 1 hour to form swollen resultant;
b) adding an aqueous solution of cationic, anionic, or hydrophobic agent to the resultant of (a) and heat the mixture to a temperature ranging from about 30° C. to 80° C. for about 1 to about 10 hours; and
c) lowering the temperature of the resultant of (b) to a room temperature, neutralizing the resultant with acid, and washing with different strength of IPA, acetone, or methanol; and
d) drying the resultant of step (c) in a temperature ranging from about 30° C. to about 80° C. to retrieve functionalized dihydroxyalkyl cellulose comprising cationic, anionic, or hydrophobic moieties.

18. A method for preparing hydrophobic functional dihydroxyalkyl cellulose. comprising the steps of:
a) mixing from about 5% (w/w) to 45% (w/w) of dihydroxyalkyl cellulose, from about 20% (w/w) to 70% (w/w) of IPA, from about 5% (w/w) to 50% (w/w) of water and from about 1% (w/w) to 15% (w/w) of caustic in a flask and stirring from about 0.1 hour to 1 hour;
b) adding from about 1% (w/w) to 30% (w/w) of cationic, anionic, or hydrophobic agent to the resultant of (a) and heat the mixture to a temperature ranging from about 30° C. to 120° C. for about 1 to about 16 hours; and
c) lower the temperature of the resultant of (b) to room temperature, neutralize the resultant of step (b) with 50% (w/w) to 100% (w/w) of acid, and wash with different strength of 50% (w/w) to 100% (w/w) of IPA, acetone, or methanol; and
d) dry the resultant of step (c) in temperature ranging from about 30° C. to about 80° C. in an oven to retrieve cationic, anionic, or hydrophobic functional dihydroxyalkyl cellulose.

19. The method according to claim 16(a), wherein the cellulose is selected from the group consisting of wood pulps, and cotton linters.

20. The method according to claim 16, wherein the caustic solution is prepared from the group consisting of sodium hydroxide, potassium hydroxide, calcium hydroxide, sodium carbonate, potassium carbonate, sodium bicarbonate, potassium bicarbonate and combinations thereof.

21. The method according to claim 16, wherein the diol compound is selected from the group consisting of oxiranylalkanol, glycerol monohalohydrin and combinations thereof.

22. The method according to claim 21, wherein the glycerol monohalohydrin is selected from the group consisting of 3-chloro-1,2-propanediol, 3-bromo-1,2-dihydroxypropanediol, 3-iodo-1,2-dihydroxypropanediol, 3-tosyl-1,2-dihydroxypropanediol, and combinations thereof.

23. The method according to claim 21, wherein the oxiranylalkanol compound is selected from the group consisting of oxiranylmethanol (glycidol), oxiranylethanol, oxiranylpropanol, oxanylbutanol, oxanylpentanol, 2-(1-Ethoxyethoxy)-2-(2-oxiranyl) ethanol, and combinations thereof.

24. A personal care composition comprising:
a) from about 0.1 to about 20% (w/w) water-soluble, partially biodegradable dihydroxyalkyl cellulose of claim 1, and
b) from about 80 to about 99.9% (w/w) of one or more personal care ingredient.

25. The personal composition according to claim 24, wherein the composition is a hair care composition, a skin care composition, a shampoo composition, an oral care composition or a bodywash composition.

26. The personal composition according to claim 24, wherein the personal care ingredient is selected from the group consisting of surfactants, fatty acid soap, hair and skin conditioning agents, suspending aids, emollients, emulsifiers, rheology modifiers, thickening agents, hair growth promoters, self-tanning agents, sunscreens, skin lighteners, anti-aging compounds, anti-wrinkle compounds, anti-cellulite compounds, anti-acne compounds, anti-dandruff agents, antiperspirant agents, deodorant agents, hair fixatives, particulates, abrasives, moisturizers, antioxidants, keratolytic agents, anti-static agents, foam boosters, hydrotropes, solublizing agents, chelating agents, pH adjusting agents, chelating agents, buffering agents, botanicals, hair colorants, hair dye, oxidizing agents, reducing agents, hair and skin bleaching agents, pigments, anticaries, anti-tartar agents, anti-plaque agents, solvents, a rheology modifier, and combinations thereof.

27. A hair care composition comprising:
a) from about 0.1 to about 20% (w/w) water-soluble, partially biodegradable dihydroxyalkyl cellulose of claim 1, and
b) from about 80 to about 99.9% (w/w) of one or more hair care ingredient.

28. A bodywash composition comprising:
a) from about 0.1 to about 10% (w/w) water-soluble, partially biodegradable dihydroxyalkyl cellulose of claim 1, and
b) from about 90 to about 99.9% (w/w) of one or more ingredient.
